(19) United States
(12) Patent Application Publication
(10) Pub. No.: US 2017/0189366 A1
(43) Pub. Date: Jul. 6, 2017

(54) METHOD FOR TREATING DISORDERS RELATED TO COMPLEMENT ACTIVATION

(71) Applicant: CASE WESTERN RESERVE UNIVERSITY, Cleveland, OH (US)

(72) Inventors: M. Edward Medof, Cleveland, OH (US); Feng Lin, Cleveland, OH (US); Qing Li, Cleveland, OH (US)

(21) Appl. No.: 15/339,367

(22) Filed: Oct. 31, 2016

Related U.S. Application Data
(63) Continuation of application No. 12/514,024, filed on May 7, 2009, now abandoned.
(60) Provisional application No. 60/857,259, filed on Nov. 7, 2006.

(51) Int. Cl. A61K 31/245; A61K 31/24

(52) U.S. Cl. CPC A61K 31/245; A61K 31/24

(57) ABSTRACT

A method for treating or preventing a complement activated T-cell mediated disorder in a subject includes administering a therapeutically effective amount of a pharmaceutical composition to the subject. The pharmaceutical composition includes at least one amidine compound or pharmaceutically acceptable salt.

### RELATED APPLICATION

This application claims priority from U.S. Provisional Application No. 60/857,259, filed Nov. 7, 2006, the subject matter, which is incorporated herein by reference.

### GOVERNMENT FUNDING

This invention was made with government support under Grant No. NIH RO1 AI23598 and R01 NS052471 awarded by the National Institutes of Health. The United States government has certain rights in the invention.

### TECHNICAL FIELD

The present invention relates generally to a method for treating or preventing a complement mediated disorders, and more particularly to a method for treating or preventing T cell autoreactivity in autoimmune disease associated with complement activation and/or complement activation mediated disorders.

### BACKGROUND OF THE INVENTION

T-cell mediated diseases represent a large number of immune system disorders. In particular, T-cells are thought to be the cells that start and perpetuate autoimmune diseases. Autoimmune diseases are a group of eighty serious, chronic illnesses that afflict millions of people in the United States alone. Autoimmune diseases are characterized by reactivity of the immune system to endogenous (self) antigens. These immune responses to self antigens are maintained by the persistent or recurrent activation of self-reactive T-cells and, directly or indirectly, the self-reactive T-cells are responsible for the characteristic tissue injury and destruction seen in autoimmune diseases. Although many treatments for autoimmune diseases and other T-cell mediated diseases have been proposed, there is still a need for additional treatments.

Ophthalmic diseases may lead to vision loss and can include front-of-eye diseases such as corneal edema, anterior uveitis, pterygium, corneal diseases or opacifications with an exudative or inflammatory component, and conjunctivitis, as well as back-of-eye diseases such as exudative macular degeneration, macular edema arising from laser treatment of the retina, diabetic retinopathy, and age-related macular degeneration (AMD). Back-of-eye diseases comprise the largest number of causes for vision loss in the developed world.

Despite the prevalence of AMD and the tremendous morbidity associated with it both medically and financially, the underlying cause of the disease has remained obscure and no specific therapy is available to either reverse retinal changes or prevent further progression to complete blindness. Existing therapies for AMD, such as AVASTIN and laser therapy, require intravitreal injections and can result in inflammation leading to further edema (respectively). Such therapies are mostly palliative, thus allowing the disease to continue and progress with re-development of neovascularization and destruction of the macula.

### SUMMARY OF THE INVENTION

The present invention relates to a method for treating disorders associated with intraoccular complement activation or T cell autoreactivity in a subject. The method includes administering a therapeutically effective amount of a pharmaceutical composition to the subject. The pharmaceutical composition includes at least one amidine compound or pharmaceutically acceptable salt thereof having anti-complement activity. In an aspect of the invention, the amidine compound can have the following general formula (I):

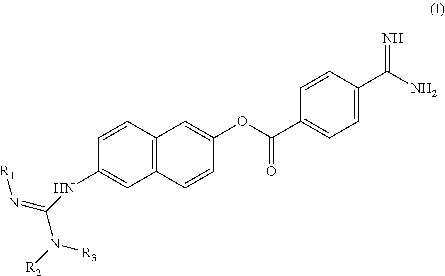

wherein R₁ and R₂ each represent a hydrogen atom, or a straight or branched chain alkyl group of 1 to 6 carbons;

wherein R₃ represents a hydrogen, a straight or branched chain alkyl group of 1 to 6 carbon atoms or a group of the formula R₄—B—(CH₂)ₙ— where

n is 1 to 2,

B is —O— or —NH— and

R₄ is a hydrogen atom, R₅—CO— or

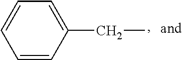

R₅ is a straight or branched chain alkyl group of 1 to 15 carbon atoms;

and wherein R₁ and R₃ taken together via 2 to 4 carbon atoms optionally form a ring containing double bonds and straight or branched alkyl groups of 1 to 4 carbon atoms as substituents or a pharmaceutically acceptable salt thereof. In another aspect of the invention, the amidine compound can have the formula (II):

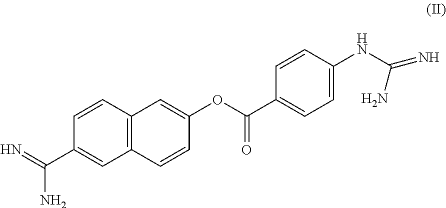 [SMILES: N=C(N)Nc1ccc(C(=O)Oc2ccc3cc(C(=N)N)ccc3c2)cc1]

or a pharmaceutically acceptable salt thereof.

Another aspect of the present invention relates to a method of treating T cell mediated autoimmune disorders in a subject. The method includes administering a therapeutically effective amount of a pharmaceutical composition to the subject. The pharmaceutical composition includes at least one amidine compound or pharmaceutically acceptable salt thereof having anti-complement activity. In an aspect of the invention, the amidine compound can have the following general formula (I):

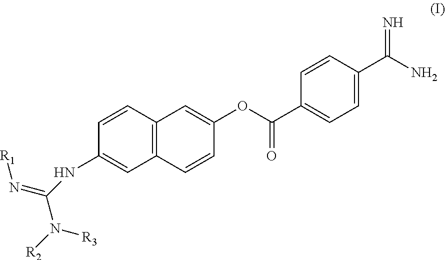

wherein R₁ and R₂ each represent a hydrogen atom, or a straight or branched chain alkyl group of 1 to 6 carbons;

wherein R₃ represents a hydrogen, a straight or branched chain alkyl group of 1 to 6 carbon atoms or a group of the formula R₄—B—(CH₂)ₙ— where

n is 1 to 2,

B is —O— or —NH— and

R₄ is a hydrogen atom, R₅—CO— or

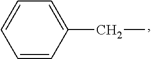

and

R₅ is a straight or branched chain alkyl group of 1 to 15 carbon atoms;

and wherein R₁ and R₃ taken together via 2 to 4 carbon atoms optionally form a ring containing double bonds and straight or branched alkyl groups of 1 to 4 carbon atoms as substituents or a pharmaceutically acceptable salt thereof. In another aspect of the invention, the amidine compound can have the formula (II):

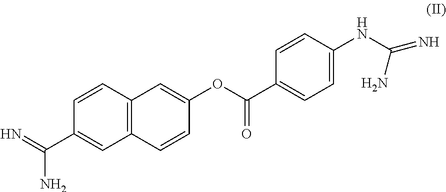 [SMILES: N=C(N)Nc1ccc(C(=O)Oc2ccc3cc(C(=N)N)ccc3c2)cc1]

or a pharmaceutically acceptable salt thereof.

A further aspect of the present invention relates to a method of treating retinal or choroidal degenerative disease in a subject. The method includes administering a therapeutically effective amount of a pharmaceutical composition to the subject. The pharmaceutical composition includes at least one amidine compound or pharmaceutically acceptable salt thereof having anti-complement activity. In an aspect of the invention, the amidine compound can have the following general formula (I):

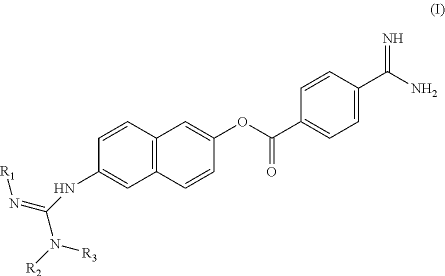

wherein R₁ and R₂ each represent a hydrogen atom, or a straight or branched chain alkyl group of 1 to 6 carbons;

wherein R₃ represents a hydrogen, a straight or branched chain alkyl group of 1 to 6 carbon atoms or a group of the formula R₄—B—(CH₂)ₙ— where

n is 1 to 2,

B is —O— or —NH— and

R₄ is a hydrogen atom, R₅—CO— or

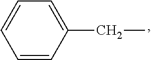

and

R₅ is a straight or branched chain alkyl group of 1 to 15 carbon atoms;

and wherein R₁ and R₃ taken together via 2 to 4 carbon atoms optionally form a ring containing double bonds and straight or branched alkyl groups of 1 to 4 carbon atoms as substituents or a pharmaceutically acceptable salt thereof. In another aspect of the invention, the amidine compound can have the formula (II):

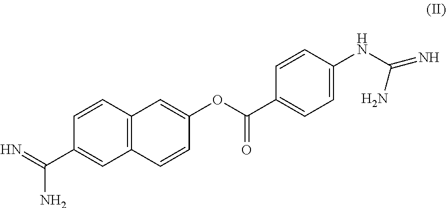 [SMILES: N=C(N)Nc1ccc(C(=O)Oc2ccc3cc(C(=N)N)ccc3c2)cc1]

or a pharmaceutically acceptable salt thereof.

### BRIEF DESCRIPTION OF THE DRAWINGS

The foregoing and other features of the present invention will become apparent to those skilled in the art to which the present invention relates upon reading the following description with reference to the accompanying drawings, in which:

FIGS. 1A-C are a series of graphs showing the ability of an amidine compound according to the present invention to inhibit alternative pathway-dependent amplification of C3 cleavage. In FIG. 1A, factor B, factor D, and C3 were mixed in the absence or presence of increasing concentrations of the amidine compound and generation of C3a was quantified by ELISA. In FIG. 1B, factor B, factor D, C3, and a limiting amount of factor H were mixed in the absence or presence of two doses of the amidine compound following initial deposition of C3b on Bruch's membrane via anti-CEP initiated classical pathway activation. In FIG. 1C, factor B, factor D, C3, and a limiting amount of factor H were mixed in the absence or presence of two doses of the amidine compound following initial deposition of C3b on the RPE-43 cell line by anti-class I monomorphic antibody; and

FIGS. 2A-B are a series of flat mounts showing the effects on lesion size of the amidine compound in a laser-induced CNV mouse model. FIG. 2A shows representative flat mounts of the RPE-Bruch's membrane-choroid complex in control-treated mice. FIG. 2B shows representative flat mounts of the RPE-Bruch's membrane-choroid complex of mice treated with the amidine compound.

FIG. 3 is a graph illustrating in vivo inhibitory effect of FUT-175 in an EAE mouse model.

### DETAILED DESCRIPTION

One aspect of the present invention relates to a method of treating T-cell mediated diseases and/or T-cell autoreactivity in autoimmune diseases. “Treat” is used herein to mean to reduce (wholly or partially) the symptoms, duration or severity of a disease, including curing the disease, or to prevent the disease.

T-cell mediated diseases can include graft rejection, graft versus host disease, unwanted delayed-type hypersensitivity reactions (such as delayed-type allergic reactions), T-cell mediated pulmonary diseases, and autoimmune diseases. T-cell mediated pulmonary diseases include sarcoidosis, hypersensitivity pneumonitis, acute interstitial pneumonitis, alveolitis, pulmonary fibrosis, idiopathic pulmonary fibrosis and other diseases characterized by inflammatory lung damage. Autoimmune diseases include multiple sclerosis, neuritis, polymyositis, psoriasis, vitiligo, Sjogren's syndrome, rheumatoid arthritis, Type 1 diabetes, inflammatory bowel diseases (e.g., Crohn's disease and ulcerative colitis), celiac disease, glomerulonephritis, scleroderma, sarcoidosis, autoimmune thyroid diseases (e.g., Hashimoto's thyroiditis and Graves disease), myasthenia gravis, Addison's disease, autoimmune uveoretinitis, pemphigus vulgaris, primary biliary cirrhosis, pernicious anemia, and systemic lupus erythematosis.

It was found that complement activation of T-cells can contribute to the pathology of T-cell mediated diseases and that amidine compounds, which exhibit anti-complement activity can be systemically administered to animals with a T-cell mediated disease, such as multiple sclerosis, to inhibit, reduce, ameliorate T-cell autoreativity and activation and treat the T-cell mediated disease or autoimmune disease.

In an aspect of the invention, the T-cell mediated disease or autoimmune diseases can therefore be treated by administering to an animal or subject in need thereof an effective amount of an amidine compound or pharmaceutically acceptable salt thereof that exhibits anti-complement activity. Amidine compounds that exhibit anti-complement activity and that can be used in the therapeutic method of the present invention include, for example, amidine compounds disclosed and claimed in U.S. Pat. Nos. 4,514,416; 4,454,338; 4,570,006; 4,777,182; 5,514,713; and 5,932,603, all of which are hereby incorporated by reference in their entireties.

In an aspect of the invention the at least one amidine compound can have the following general formula (I):

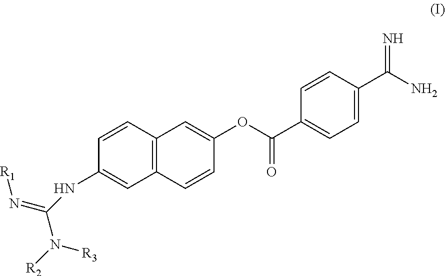

wherein R₁ and R₂ each represent a hydrogen atom, or a straight or branched chain alkyl group of 1 to 6 carbons;

wherein R₃ represents a hydrogen, a straight or branched chain alkyl group of 1 to 6 carbon atoms or a group of the formula R₄—B—(CH₂)ₙ— where

n is 1 to 2,

B is —O— or —NH— and

R₄ is a hydrogen atom, R₅—CO— or

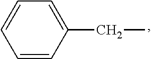

and

R₅ is a straight or branched chain alkyl group of 1 to 15 carbon atoms; And R1 and R3 taken together via 2 to 4 carbon atoms may form a ring optionally containing double bonds and straight or branched alkyl groups of 1 to 4 carbon atoms as substituents or a pharmaceutically acceptable salt thereof.

In another aspect of the invention, the at least one amidine compound can be 6-amidino-2-naphthyl-4-guanidinobenzoate, which has the following formula (II):

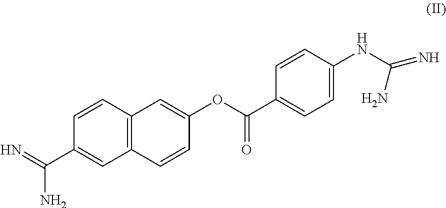 [SMILES: N=C(N)Nc1ccc(C(=O)Oc2ccc3cc(C(=N)N)ccc3c2)cc1]

or a pharmaceutically acceptable salt thereof (e.g., -amidino-2-naphthyl-4-guanidinobenzoate dimethanesulfonate).

Amidine compounds of the present invention can be provided in the form of pharmaceutical compositions. Pharmaceutical compositions can be administered to any mammal that can experience the beneficial effects of the amidine compounds of the present invention. Foremost among such mammals are humans, although the present invention is not intended to be so limited.

Pharmaceutical compositions of the present invention can be administered by any means that achieve their intended purpose. For example, administration can be by parenteral, subcutaneous, intravenous, intraarticular, intrathecal, intramuscular, intraperitoneal, or intradermal injections, or by transdermal, buccal, oromucosal, ocular routes or via inhalation. Particularly preferred is parenteral administration. The dosage administered will be dependent upon the age, health, and weight of the subject, kind of concurrent treatment, if any, frequency of treatment, and the nature of the effect desired.

When referring to an amidine compound of the present invention, applicants intend the phrase “amidine compound” to encompass not only the specified molecular entity, but also its pharmaceutically acceptable, pharmacologically active analogs, including, but not limited to, salts, esters, amides, prodrugs, conjugates, active metabolites, and other such derivatives, analogs, and related compounds.

The term “therapeutic” refers to reduction in severity and/or frequency of symptoms, elimination of symptoms and/or underlying cause, prevention of the occurrence of symptoms and/or their underlying cause, and improvement or remediation of disease. For example, treatment of a subject by administration of an amidine compound of the present invention encompasses prevention in a subject susceptible to developing a retinal or choroidal degenerative disease (e.g., at a higher risk, as a result of genetic predisposition, environmental factors, or the like) and/or in treatment of a subject having a retinal or choroidal degenerative disease.

“Effective amounts,” in terms the foregoing method, are amounts of the at least one amidine compound of formulas (I-III) effective to treat or prevent a retinal or choroidal degenerative disease in a subject.

The phrase “pharmaceutically acceptable” should be understood to mean a material which is not biologically or otherwise undesirable, i.e., the material may be incorporated into a pharmaceutical composition administered to a subject without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree.

Some of the amidine compounds disclosed herein may contain one or more asymmetric centers and may thus give rise to enantiomers, diastereomers, and other stereoisomeric forms. The present invention is also meant to encompass racemic mixtures, resolved forms and mixtures thereof, as well as the individual enantiomers that may be separated according to methods that are well know to those of ordinary skill in the art. When the amidine compounds described herein contain olefinic double bonds or other centers of geometric asymmetry, and unless specified otherwise, it is intended to include both E and Z geometric isomers.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “asymmetric center” or “chiral center” refers to a carbon atom to which four different groups are attached.

The term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule. The phrase “enantiomeric excess” refers to a mixture wherein one enantiomer is present is a greater concentration than its mirror image molecule.

In addition to the pharmacologically active compounds, the pharmaceutical compositions of the present invention can contain suitable pharmaceutically acceptable carriers comprising excipients and auxiliaries that facilitate processing of the amidine compounds into preparations that can be used pharmaceutically. The pharmaceutical compositions of the present invention may be manufactured in a manner that is, itself, known, for example, by means of conventional mixing, granulating, dragee-making, dissolving, or lyophilizing processes. Thus, pharmaceutical compositions for oral use, for example, can be obtained by combining amidine compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example, lactose or sucrose, mannitol or sorbitol, cellulose preparations and/or calcium phosphates, for example, tricalcium phosphate or calcium hydrogen phosphate, as well as binders, such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added, such as the above-mentioned starches and also carboxymethyl-starch, cross-linked polyvinyl pyrrolidone, agar, or alginic acid or a salt thereof, such as sodium alginate. Auxiliaries can include flow-regulating agents and lubricants, for example, silica, talc, stearic acid or salts thereof, such as magnesium stearate or calcium stearate, and/or polyethylene glycol. Dragee cores may be provided with suitable coatings, that if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as acetylcellulose phthalate or hydroxypropylmethylcellulose phthalate, may be used. Slow-release and prolonged-release formulations may be used with particular excipients such as methacrylic acid-ethylacrylate copolymers, methacrylic acid-ethyl acrylate copolymers, methacrylic acid-methyl methacrylate copolymers and methacrylic acid-methyl methylacrylate copolymers. Dye stuffs or pigments can be added to the tablets or dragee coatings, for example, for identification or in order to characterize combinations of active compound doses.

Other pharmaceutical compositions that can be used orally include push-fit capsules made of gelatin, as well as soft-sealed capsules made of gelatin and a plasticizer such as glycerol or sorbitol. The push-fit capsules can contain the active compounds in the form of granules that may be mixed with fillers such as lactose, binders such as starches, and/or lubricants such as talc or magnesium stearate and, optionally, stabilizers. In soft capsules, the active compounds may be dissolved or suspended in suitable liquids such as fatty oils or liquid paraffin. In addition, stabilizers may be added.

Suitable formulations for parenteral administration include aqueous solutions of the active compounds in water-soluble form, for example, water-soluble salts and alkaline solutions. Especially preferred salts can include maleate, fumarate, succinate, S,S tartrate, or R,R tartrate. In addition, suspensions of the amidine compounds as appropriate oily injection suspensions can be administered. Suitable lipophilic solvents or vehicles can include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, for example, sodium carboxymethyl cellulose, sorbitol, and/or dextran.

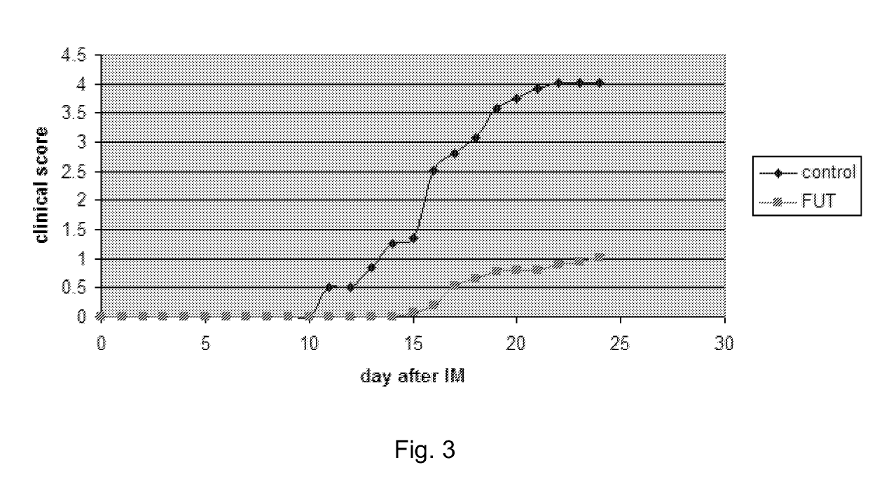

In an example of the method, a subject with a T-cell mediated autoimmune disease, such as multiple sclerosis, may be treated by administering a therapeutically effective amount of a pharmaceutical composition containing an amidine compound, such as an amidine compound having the formula (II). The pharmaceutical composition may be administered to the subject, for example, parenterally. By administering the pharmaceutical composition, complement activation of the T-cells may be reduced or inhibited. As shown in FIG. 3, which illustrate the in vivo inhibitory effect of FUT-175 in an EAE mouse, the clinical score of the EAE mice treated with FUT-175 was substantially improved with respect to the control EAE mice where no FUT-175 was administered.

Another aspect of the present invention relates generally to a method for treating or preventing a retinal or choroidal degenerative disease that is associated with intraoccular complement activation that is derived from systemic (e.g., serum or blood) and/or local (T-cell or antigen presenting cell) complement activation. The retinal or choroidal degenerative disease can include, for example, age-related macular degeneration (AMD). It was found that an amidine compound, such as 6-amidino-2-napthyl-4-guanidinobenzoate dimethanesulfonate or FUT-175, suppresses alternative pathway-dependent amplification of complement activation in vitro, and significantly reduces choroidal neovascularization in vivo. Based on this discovery, the present invention provides a method for treating or preventing a retinal or choroidal degenerative disease in a subject by administering a therapeutically effective amount of a pharmaceutical composition comprising at least one amidine compound with anti-complement activity, such as described above. In an aspect of the invention, the amidine compound can be selected from formulas (I-II) below. The at least one amidine compound may be combined or mixed with one or more other agents, such as non-toxic pharmaceutically acceptable carriers, diluents, adjuvants and/or other ingredients, if desired.

In another aspect of the present invention, a retinal or choroidal degenerative disease may be treated or prevented by administering a therapeutically effective amount of a pharmaceutical composition comprising at least one amidine compound selected from formulas (I-II). A retinal or choroidal degenerative disease may generally include diseases or conditions of the eye associated with neovascularization, including, for example, retinal and/or choroidal neovascularization. More particularly, a retinal or choroidal degenerative disease can include, without limitation, wet macular degeneration, dry macular degeneration, early-onset macular degeneration, atrophic macular degeneration, neovascular macular degeneration, AMD, choroidal neovascularization, retinal pigment epithelium detachment, atrophy of retinal pigment epithelium, Best's disease, vitelliform, Stargardt's disease, juvenile macular dystrophy, fundus flavimaculatus, Behr's disease, Sorsby's disease, Doyne's disease, honeycomb dystrophy, North Carolina macular dystrophy, pattern dystrophy, dominant drusen, malattia leventinese, chorioretinal degenerations, retinal degenerations, photoreceptor degenerations, RPE degenerations, mucopolysaccharidoses, and rod-cone dystrophies.

In an example of the method, a subject having a retinal or choroidal degenerative disease may be treated by administering a therapeutically effective amount of a pharmaceutical composition containing an amidine compound, such as having formula (II). The pharmaceutical composition may be administered to the subject parenterally, for example. By administering the pharmaceutical composition, alternative pathway-dependent amplification of complement activation may be reduced or inhibited. More particularly, administration of the pharmaceutical composition may reduce or inhibit C3b activation and, in turn, reduce or inhibit choroidal neovascularization. Treatment with the pharmaceutical composition of the present invention may reduce or prevent AMD and thus reduce or prevent progressive vision loss in the subject.

The following examples are for the purpose of illustration only and are not intended to limit the scope of the claims, which are appended hereto.

### Example 1

### Suppression of Complement Activation on RPE Cells by FUT-175

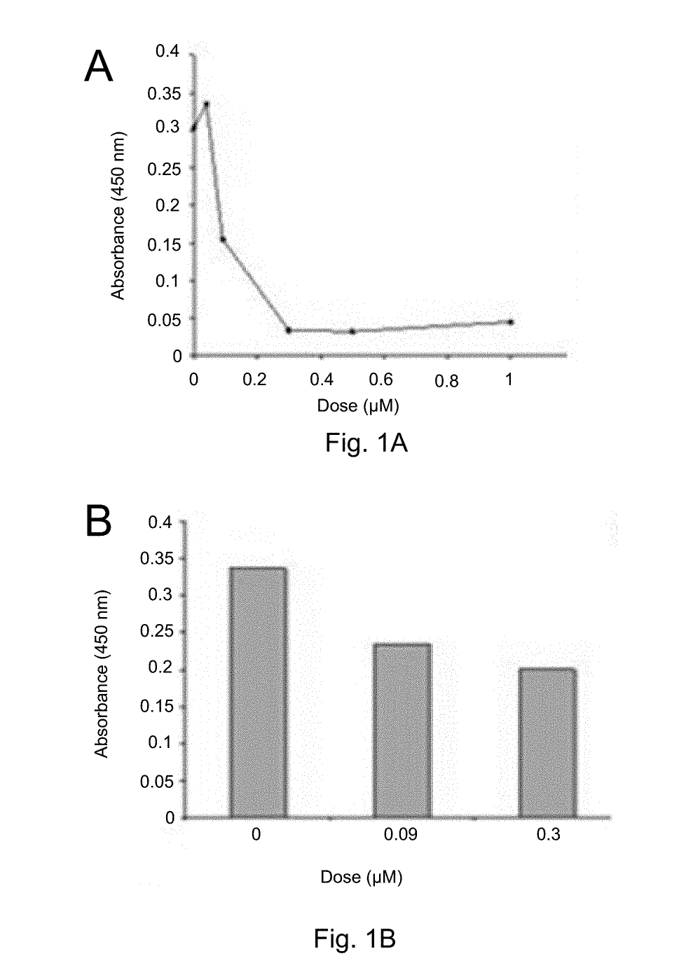

To determine how effective FUT-175 is in preventing alternative pathway-dependent amplification of complement activation on Bruch's membrane and RPE cells, we seeded purified Bruch's membrane from an AMD patient and the RPE-43 cell line with C3b (as would occur with anti-CEP autoantibodies) by sensitization with anti-CEP and anti-HLA monomorphic monoclonal antibodies, respectively, followed by incubation with purified C1, C4, C2, and decay of C1 and C2. As shown in FIGS. 1A-C, upon subsequent addition of alternative pathway components (C3, factor B, factor D, and factor H), this small molecule inhibited further C3b activation in a dose-dependent manner. About 50% inhibition was achieved in both sites at 0.3 μM, a level lower than that used clinically.

### Example 2

### Suppression of Angiogenesis in the Murine Laser-Induced CNV Model by FUT-175

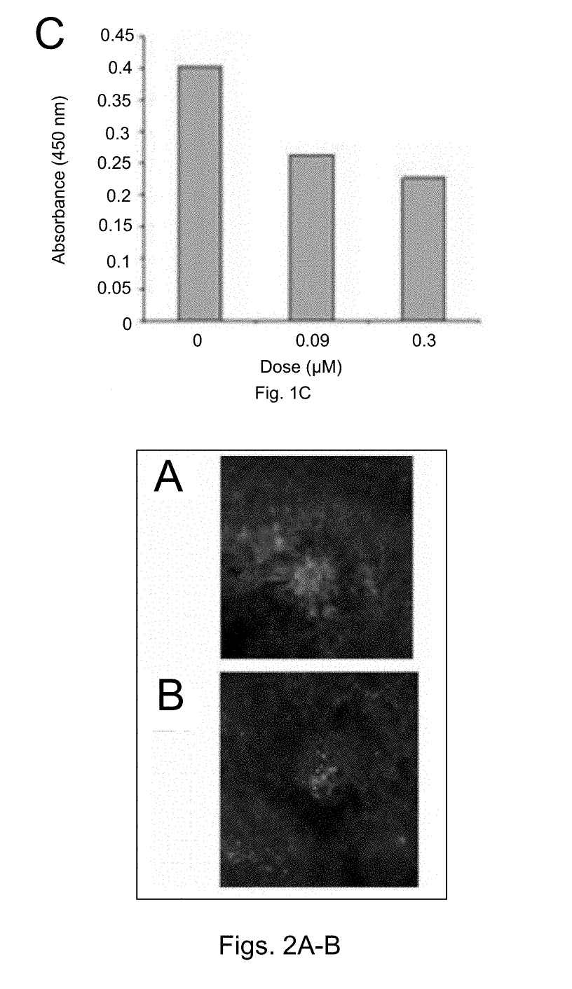

As part of our exploration of different strategies for inhibiting complement activation intraocularly so as to arrive at what methodology could be most efficacious for treating AMD patients clinically, we utilized the standard murine CNV model and analyzed the effect of an i.p. injection of FUT-175. As shown diagrammatically in FIGS. 2A-B, the drug reduced lesion size by >80%.

### Example 3

### In Vivo Inhibitory Effect of FUT-175

The in vivo inhibitory effect of FUT-175 on complement activation of T-cells was determined in Female C57BL/6 mice (n=38). The mice were divided into treatment groups and administered parenterally a control (equal volume PBS i.p (n=20)) or FUT-175 (10 mg/kg/every other day i.p from day 0 (n=18)). The results are shown in FIG. 3. FIG. 3 shows that the clinical score that the degrees of multiple sclerosis (MS) symptoms in the mice treated with FUT-175 was substantially reduced compared to control mice.

From the above description of the invention, those skilled in the art will perceive improvements, changes and modifications. Such improvements, changes and modifications within the skill of the art are intended to be covered by the appended claims. All references, patents, and publications cited herein are incorporated by reference in their entirety.

1-10. (canceled)

11. A method of treating T cell mediated autoimmune disorders in a subject, the method comprising:
administering a therapeutically effective amount of a pharmaceutical composition to the subject, the pharmaceutical composition including at least one amidine compound or pharmaceutical salt thereof having anti-complement activity, 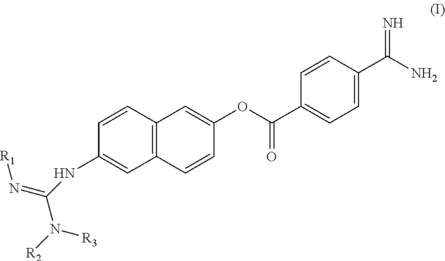
wherein the at least one amidine compound includes formula (I):
wherein R₁ and R₂ each represent a hydrogen atom, or a straight or branched chain alkyl group of 1 to 6 carbons;
wherein R₃ represents a hydrogen, a straight or branched chain alkyl group of 1 to 6 carbon atoms or a group of the formula R₄—B—(CH₂)ₙ— where
n is 1 to 2,
B is —O— or —NH— and
R₄ is a hydrogen atom, R₅—CO— or 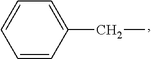
and
R₅ is a straight or branched chain alkyl group of 1 to 15 carbon atoms;
and wherein R₁ and R₃ taken together via 2 to 4 carbon atoms optionally form a ring containing double bonds and straight or branched alkyl groups of 1 to 4 carbon atoms as substituents or a pharmaceutically acceptable salt thereof.

12. (canceled)

13. The method of claim 11, the pharmaceutical composition being administered parenterally, transdermally, intranasally, sublingually, transmucosally, intra-arterially, intradermally or intravitreally.

14. The method of claim 11, the pharmaceutical composition being administered parenterally.

15. The method of claim 11, the T-cell mediated disease comprising at least one of T-cell mediated sarcoidosis, hypersensitivity pneumonitis, acute interstitial pneumonitis, alveolitis, pulmonary fibrosis, idiopathic pulmonary fibrosis, other diseases characterized by inflammatory lung damage, multiple sclerosis, neuritis, polymyositis, psoriasis, vitiligo, Sjogren's syndrome, rheumatoid arthritis, Type 1 diabetes, inflammatory bowel diseases, celiac disease, glomerulonephritis, scleroderma, sarcoidosis, autoimmune thyroid diseases, myasthenia gravis, Addison's disease, autoimmune uveoretinitis, pemphigus vulgaris, primary biliary cirrhosis, pernicious anemia, and systemic lupus erythematosis.

16. The method of claim 11, the T-cell mediated disease being multiple sclerosis.

17. The method of claim 11, wherein the amidine compound has the formula (II): 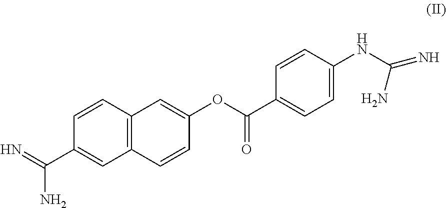 [SMILES: N=C(N)Nc1ccc(C(=O)Oc2ccc3cc(C(=N)N)ccc3c2)cc1]
or a pharmaceutically acceptable salt thereof.

18-24. (canceled)
